(19) United States
(12) Patent Application Publication
(10) Pub. No.: US 2007/0202117 A1
(43) Pub. Date: Aug. 30, 2007

(54) Compositions and Methods Of Modulating the Immune Response

(76) Inventors: Herman Groen, Groningen (NL); Al Mateczun, Silver Springs, MD (US); Gail Chapman, Rockville, MD (US); Les Baillie, Rockville, MD (US); Stanley Goldman, Rockville, MD (US); Mark Albrecht, Rockville, MD (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/615,655

(22) Filed: Dec. 22, 2006

Related U.S. Application Data
(60) Provisional application No. 60/753,539, filed on Dec. 22, 2005.

(51) Int. Cl. A61K 48/00; A61K 39/40; A61K 39/295; A61K 39/07

(52) U.S. Cl. 424/164.1; 424/246.1; 424/202.1; 514/44

(57) ABSTRACT

The present invention provides methods and compositions of enhancing the immune response to an antigen.

### RELATED APPLICATIONS

This application claims priority to U.S. Ser. No. 60/753,539 filed Dec. 22, 2005, which is incorporated herein by reference in its entirety.

### FIELD OF THE INVENTION

The invention relates to compositions and methods of protein vaccines and their use in preventing and treating infection.

### BACKGROUND OF THE INVENTION

In the generic sense, the process of artificial induction of immunity, in an effort to protect against infectious disease, works by priming the immune system with an immunogen. Stimulating immune response, via use of an infectious agent, is known as immunization. Vaccinations involve the administration of one or more immunogens, in the form of live, but weakened (attenuated) infectious agents, which normally are either weaker, but closely-related species (as with smallpox and cowpox), strains weakened by some process or recombinant proteins.

### SUMMARY OF THE INVENTION

The present invention provides methods of inducing an immune response, e.g. an antibody response, to an antigen in a subject by administering to a subject an antigen and an antibody or fragment thereof specific for the antigen. Optionally, two, three, four, five or more antibodies specific for the antigen are administered. Preferably each antibody is specific for a different epitope on the antigen. The immune response is of a higher magnitude, e.g. higher titer, than when the antigen is administered without the antibody.

Also included in the invention are methods of enhancing antigen presentation of an antigen by contacting an antigen presenting cell with an antigen and an antibody or fragment thereof specific for the antigen. Antigen presenting cells include for example macrophages, B-lymphocytes, and all cells expressing MHC class II and or class I. The cell is contacted in vitro, in vivo or ex vivo.

In a further aspect the invention provides methods of increasing the time an antigen is in circulation comprising administering to a subject an antigen and an antibody or fragment thereof specific for the antigen. By increase is meant that the antigen is in circulation longer compared to an antigen that is administered without the antibody. The increase is 2, 3, 4, 5, 10 or more fold.

The antibody and the antigen are administered concurrently or sequentially. For example, the antibody is administered prior to or after administration of the antigen.

Optionally the antibody and the antigen is administered post exposure to a pathogen such as anthrax.

The antigen is any compound to which an immune response is desired. For example the antigen is a pathogen or immunogenic component thereof. The pathogen is a toxin, a virus, a bacterium, a fungus, a protozoan, a mycloplasma, a rickettsia or a parasite. In some embodiments the antigen is Bacillus anthracis or component thereof such as a Bacillus anthracis protective antigen polypeptide or a Bacillus anthracis lethal factor polypeptide. Preferably, the antigen is administered in a form of a vaccine such as the anthrax vaccine AVA. The vaccine is a protein based vaccine or a DNA based vaccine.

The antibody is a monoclonal or polyclonal antibody or fragment thereof. Optionally the antibody is fully human or humanized. In some embodiments the antibody is a anti-anthrax antibody. Exemplary anti-anthrax antibodies include IQNPA or IQNLF.

Also included in the invention are compositions containing IQNPA and/or IQNLF and an anthrax vaccine such as AVA.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a graph showing the survival of female A/J mice passively protected with a single does of IQNPA-2 or IQNLF-1 prior to challenge with Anthrax.

FIG. 2 is a bar graph showing anti-PA IgG titer after B. anthracis Sterne strain spore challenge. Anti-PA IgG titers increases after initial challenge and were the greatest in mice treated with IQNPA-1.

FIG. 3 is a bar graph showing anti-LF IgG titer after B. anthracis Sterne strain spore challenge. Anti-PA IgG titers increases after initial challenge and were the greatest in mice treated with IQNLF-1.

FIG. 4 is a bar graph of the clearance of antibodies from the mice treated with IQNPA-2 or IQNLF-1.

### DETAILED DESCRIPTION OF THE INVENTION

The present invention is based on the observation that human monoclonal antibodies directed against the two components of the anthrax lethal toxin, protective antigen (PA) and lethal factor (LF), were able to passively protect naive mice against challenge with anthrax spores while at the same time promoting the stimulation of a protective immune response by the infected animal. It was surprising that the PA and LF specific human monoclonal antibody enhanced the mouse produced antibody response to PA and LF respectively.

These results demonstrate the feasibility of developing post-exposure therapeutics based on a combination of antibodies and a vaccine. This approach would provide real time protection while at the same time stimulating the adaptive immune response to confer long-term immunity. These findings have wide ranging implications not only for the treatment of anthrax but also for the field of vaccination in general, demonstrating that the presence of antibodies with specificity for a vaccine can enhance the magnitude of the subsequent immune response.

Passive antibody administration at the time of immunization is known to initiate a complex series of events, which typically result in the suppression of more than 99% of primary humoral responses. Not to be bound by theory, it is hypothesized that monoclonal antibodies when given passively bind to their corresponding anthrax toxin components forming an antibody complex which enhances antigen presentation via Fc receptors located on the surface of antigen presenting cells (APCs). Dendritic cells and macrophages are the major APCs in the immune system and are involved in the activation and differentiation of CD4+ and CD8+ T cells. Antigens internalized through specific membrane receptors such as surface Ig and Fc receptors are more efficiently presented to CD4+ T cells than is the case with the soluble form of the antigen particularly in respect to MHC class II-restricted epitopes (Hamano et al., 2000). Passively administered monoclonals to PA and LF bind to anthrax toxin produced by the infecting bacterium and prevent it from killing the animal and allow for more time for the subject to generate an immune response. The antibody/toxin complex then binds to Fc receptors on APCs triggering cell activation, phagocytosis and subsequently enhancing Ag presentation to CD4+ T cells, which have been shown to be important in mediating strong antibody and memory responses (e.g., secondary). The immune response to PA is known to be T cell dependent (Musson et al., 2003) the immune response to LF. The enhanced antibody response seen following the second challenge could also be due in part to the presence of compliment C3 products (Baiu et al., 1999). Cross linking of the compliment and antigen receptors on B cells lowers the threshold of B cell activation. In unpublished studies we have shown that a PA-C3d fusion protein enhanced the antibody response to PA. Thus binding of C3 to the antigen-antibody complex may further enhance the magnitude of the resulting immune response.

To date, antibodies with specificity for both subunits of lethal toxin, Protective Antigen (PA) and Lethal Factor (LF), have been isolated. Administration of multiple human monoclonal antibodies with specificity for PA and LF not only maximizes the therapeutic window (by hitting the toxin at two different sites), but will be of particular value in the event of an attack with strains which have been genetically engineered to circumvent key epitope binding sites and which may be resistant to antibiotics.

Accordingly, the invention features methods of inducing an immune response (e.g., primary or secondary) to an antigen by administering to subject a composition containing an antigen and an antibody or fragment thereof specific for the antigen. Additionally, antigen presentation is enhanced by an antigen presenting cell with contacting an antigen and an antibody or fragment thereof specific for the antigen. Also included in the invention are vaccine compositions including an antigen and an antibody specific for the antigen. Optionally, the antigen is in the form of a vaccine.

The composition and methods of the invention can be used to prevent or treat, i.e., cure, ameliorate, lessen the severity of, or prevent or reduce contagion of viral, bacterial, fungal, and parasitic infectious diseases, cancer, as well as to treat allergies.

The compositions are useful in methods of inducing an immune response to the antigen in a subject, such as a human, or an animal such as a dog, cat, sheep, horse, cow, or pig. (i.e., immunization).

As used herein, the following definitions are supplied in order to facilitate the understanding of this case. To the extent that the definitions vary from meanings known to those skilled in the art, the definitions below control.

By “biological component” is meant any compound created by or associated with a cell, tissue, bacteria, virus, or other biological entity, including peptides, proteins, lipids, carbohydrates, hormones, or combinations thereof.

By “adjuvant compound” is meant any compound that increases an immunogenic response or the immunogenicity of an antigen or vaccine.

By “antigen” is meant any compound capable of inducing an immune response. Antigen Antigens are proteins, carbohydrates or lipids. Exemplary antigens include, toxins, bacteria, fungi, protozia, mycoplasma, parasites, rickettsia, foreign blood cells, cancer cells and the cells of transplanted organs. Preferably, the antigen is Anthrax, Hepatitis C, HIV, Hepatitis B, Papilloma virus, Malaria, Tuberculosis, Herpes Simplex Virus, Chlamydia, and Influenza, or a biological component thereof, for example, a viral, bacterial or other polypeptide.

By “immunoglobulin” is meant a any polypeptide or protein complex that is secreted by B-cells or B-cell fusions and that functions as an antibody in the immune response by binding with a specific antigen. Immunoglobulins as used herein include IgA, IgD, IgE, IgG, and IgM. Regions of immunoglobulins include the Fc region and the Fab region, as well as the heavy chain or light chain immunoglobulins.

By “antigen presentation” is meant the expression of an antigen on the surface of a cell in association with one or more major hisocompatability complex class I or class II molecules. Antigen presentation is measured by methods known in the art. For example, antigen presentation is measured using an in vitro cellular assay as described in Gillis, et al., J. Immunol. 120: 2027 1978.

By “immunogenicity” is meant the ability of a substance to stimulate an immune response. Immunogenicity is measured, for example, by determining the presence of antibodies specific for the substance. The presence of antibodies is detected by methods know in the art, for example, an ELISA assay.

By “immune response” is meant a cellular activity induced by an antigen, such as production of antibodies or presentation of antigens or antigen fragments. The immune response can be divided into several phases—the “innate” first response, mediated by cells able to destroy and phagocytose (engulf) a large range of foreign organisms; the secondary, “adaptive” response, characterized by the generation of antibodies and T cells that are specific for the antigen; and a third, “suppression” phase, where the production of immune cells reverts to normal (homeostasis), and the information necessary to mount a future immune response to that antigen is retained in bone marrow memory cells.

By “proteolytic degradation” is meant degradation of the polypeptide by hydrolysis of the peptide bonds. No particular length is implied by the term “peptide.” Proteolytic degradation is measured, for example, using gel electrophoresis.

The “cell” includes any cell capable of antigen presentation. For example, the cell is a somatic cell, a B-cell, a macrophage or a dendritic cell.

Methods of Modulating the Immune Response

An immune response is induced or the time in which an antigen is in circulation is increased in a subject by administering a subject an antigen and an antibody or fragment thereof specific for the antigen. One, two, three, four, five or more antibodies specific for a different epitope on the antigen are administered. The subject is a mammal such as human, a primate, mouse, rat, dog, cat, cow, horse, or pig. The immune response is humoral or cellular. By induced it is meant to bring about or stimulate the occurrence of an immune response. An immune response is measured by methods known in the art such as antibody production.

The immune response is of a higher magnitude then when the antigen is administered alone. By higher magnitude is meant that the immune response produces a greater amount of antigen specific antibody (e.g., higher titer), antibodies with higher affinity for the antigen, increases activation and expansion of T-cells or increases cytokine production. Increased antibody production, secretion and/or affinity is measured by methods known to those of ordinary skill in the art, including ELISA, the precipitin reaction, and agglutination reactions.

Antigen presentation is enhanced by contacting an antigen presenting cell with a antigen and an antibody or fragment thereof specific for the antigen. Antigen presenting cells include macrophages, B-lymphocytes, and all cells expressing MHC class II and or class I. Antigen presentation is the expression antigen on surface of a cell in a form recognizable by lymphocytes. Antigen presentation is determined by methods known in the art such as measuring IFN gamma production, IL-2 production or MHC class I or II and or CD80 or CD86 expression.

In some embodiments the antigen and antibody are administered to the subject or the cell is contacted simultaneously. Alternatively, the antigen is administered to the subject or the cell is contacted prior to or after the antibody. The cell is contacted in vivo, in vitro or ex vivo.

An antigen includes any compound, cell or tissue to which an immune response is desired. An antigen includes any substance that, when introduced into the body, stimulates an immune response, such as the production of an antibody from a B cell, activation and expansion of T cells, and cytokine expression (e.g., interleukins). By a “B cell” or “B lymphocyte” it is meant an immune cell that is responsible for the production of antibodies. By a “T cell” or “T lymphocyte” it is meant a member of a class of lymphocytes, further defined as cytotoxic T cells, helper T cells and regulatory T-cells. T cells regulate and coordinate the overall immune response, identifying the epitopes that mark the antigens, and attacking and destroying the diseased cells they recognize as foreign, or offering help for the induction of cells that attack and destroy or produce antibody.

Examples of viral antigens include, but are not limited to, adenovirus polypeptides, alphavirus polypeptides, calicivirus polypeptides, e.g., a calicivirus capsid antigen, coronavirus polypeptides, distemper virus polypeptides, Ebola virus polypeptides, enterovirus polypeptides, flavivirus polypeptides, hepatitis virus (AE) polypeptides, e.g., a hepatitis B core or surface antigen, herpesvirus polypeptides, e.g., a herpes simplex virus or varicella zoster virus glycoprotein, immunodeficiency virus polypeptides, e.g., the human immunodeficiency virus envelope or protease, infectious peritonitis virus polypeptides, influenza virus polypeptides, e.g., an influenza A hemagglutinin, neuramimidase, or nucleoprotein, leukemia virus polypeptides, Marburg virus polypeptides, orthomyxovirus polypeptides, papilloma virus polypeptides, parainfluenza virus polypeptides, e.g., the hemagglutinin/neuramimidase, paramyxovirus polypeptides, parvovirus polypeptides, pestivirus polypeptides, picoma virus polypeptides, e.g., a poliovirus capsid polypeptide, pox virus polypeptides, e.g., a vaccinia virus polypeptide, rabies virus polypeptides, e.g., a rabies virus glycoprotein G, reovirus polypeptides, retrovirus polypeptides, and rotavirus polypeptides.

Examples of bacterial antigens include, but are not limited to, Actinomyces polypeptides, Bacillus polypeptides, Bacteroides polypeptides, Bordetella polypeptides, Bartonella polypeptides, Borrelia polypeptides, e.g., B. burgdorferi OspA, Brucella polypeptides, Campylobacter polypeptides, Capnocytophaga polypeptides, Chlamydia polypeptides, Clostridium polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides, e.g., H. influenzae type b outer membrane protein, Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides, Proteus polypeptides, Pseudomonas polypeptides, Rickettsia polypeptides, Rochalimaea polypeptides, Salmonella polypeptides, Shigella polypeptides, Staphylococcus polypeptides, Streptococcus polypeptides, e.g., S. pyogenes M proteins, Treponema polypeptides, and Yersinia polypeptides, e.g., Y. pestis F1 and V antigens.

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides, e.g., P. falciparum circumsporozoite (PfCSP), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyrne polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides.

Examples of ectoparasite antigens include, but are not limited to, polypeptides (including protective antigens as well as allergens) from fleas; ticks, including hard ticks and soft ticks, flies, such as midges, mosquitos, sand flies, black flies, horse flies, horn flies, deer flies, tsetse flies, stable flies, myiasis-causing flies and biting gnats; ants; spiders, lice; mites; and true bugs, such as bed bugs and kissing bugs.

Examples of tumor-associated antigens include, but are not limited to, tumor-specific immunoglobulin variable regions, GM2, Tn, sTn, Thompson-Friedenreich antigen (TF), Globo H, Le(y), MUC1, MUC2, MUC3, MUC4, MUC5AC, MUC5B, MUC7, carcinoembryonic antigens, beta chain of human chorionic gonadotropin (hCG beta), HER2/neu, PSMA, EGFRvIII, KSA, PSA, PSCA, GP100, MAGE 1, MAGE 2, TRP 1, TRP 2, tyrosinase, MART-1, PAP, CEA, BAGE, MAGE, RAGE, and related proteins.

The antigen may be a administered in the form of a vaccine. The vaccine is a protein based vaccine or a DNA based vaccine. For example, the vaccine is a commercially available vaccine. Commercially available vaccines are known to those skilled in the art. Exemplary commercial vaccine include Adenovirus, Anthrax, Argentine hemorrhagic fever, BCG, Botulism antitoxin, Cholera—injectable, Cholera—oral, Cytomegalovirus immunoglobulin, Diphtheria, Diphtheria antitoxin, DT, DTaP, DTP, Eastern equine encephalitis, Gas gangrene antitoxin, H. influenzae (HbOC-DTP or -DTaP), Haemophilus influenzae (HbOC), Haemophilus influenzae (PRP-D), Haemophilus influenzae (PRP-OMP), Haemophilus influenzae (PRP-T), Hantavirus [old world], Hepatitis A, Hepatitis A+Hepatitis B, Hepatitis B, Hepatitis B+Haemoph. influenzae, Hepatitis B immune globulin, Herpes zoster, Human papillomavirus, Immune globulin, Influenza—inactivated, Influenza—live, Japanese encephalitis, Kyasanur Forest disease, Lyme disease, Measles, Measles-Mumps-Rubella, Measles-Rubella, Meningococcal, Mumps, Plague, Pneumococcal, Pneumococcal conjugate, Poliomyelitis—injectable, Poliomyelitis—oral, Q fever, Rabies, Rabies immune globulin, Rift Valley fever, Rotavirus, RSV immune globulin, Rubella, Rubella—Mumps, Smallpox, Td, Tetanus, Tetanus immune globulin, Tick-borne encephalitis, Tick-borne encephalitis globulin, Tularemia, Typhoid—injectable, Typhoid—oral, Vaccinia immune globulin, Varicella, Varicella-Zoster immune globulin, Venezuelan equine encephalitis, Western equine encephalitis or Yellow fever vaccine. Other commercially available vaccines useful in the methods of the invention includes those listed in Gideon's vaccine list.

For example, the vaccine is an Anthrax vaccine such as AVA or CAMR.

Optionally, the antigen is linked to one or more additional moieties. For example, the antigen moiety may additionally be linked to a GST fusion protein in which the mucin-Ig fusion protein sequences are fused to the C-terminus of the GST (i.e., glutathione S-transferase) sequences. Such fusion proteins can facilitate the purification of the antigen polypeptide.

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an Fab expression library. In general, an antibody molecule relates to any of the classes IgG, IgM, IgA, IgE and IgD, which differ from one another by the nature of the heavy chain present in the molecule. Certain classes have subclasses as well, such as IgG₁, IgG₂, and others. Furthermore, the light chain may be a kappa chain or a lambda chain. Reference herein to antibodies includes a reference to all such classes, subclasses and types of antibody species.

Antibodies that immunospecifically bind the antigen are prepared using standard techniques for polyclonal and monoclonal antibody preparation. The full-length antigen can be used or, alternatively, the invention provides antigenic fragments of the antigen for use as immunogens. Exemplary antibodies include anti-anthrax antibodies such as those described in WO05120567 (hereby incorporated by reference).

Any antibody can be used regardless of the method used to generate the antibody. Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies against carbohydrate moieties, various suitable host animals (e.g., rabbit, goat, mouse, fish, birds or other mammal) may be immunized by one or more injections with the native protein carrying a carbohydrate moiety, a synthetic variant thereof, or a derivative of the foregoing.

Furthermore, the carbohydrate may be conjugated to a protein known to be immunogenic in the mammal being immunized. Examples of such immunogenic proteins to which the carbohydrate moiety is attached include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate) and CpG dinucleotide motifs (Krieg, A. M. Biochim Biophys Acta 1489(1):107-16, 1999). In some aspects of the invention it is not necessary to immunize a subject with the carbohydrate to produce an anti-carbohydrate antibody, for example, the carbohydrate antibody may be a pre-formed naturally occurring antibody that is already present in the subject's blood.

The polyclonal antibody molecules directed against the carbohydrate moiety can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the carbohydrate moiety and are characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the carbohydrate moiety. Alternatively, the lymphocytes can be immunized in vitro.

Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the carbohydrate moiety can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771%. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

Pharmaceutical Compositions

The antigen and anti-antigen antibodies can be formulated in pharmaceutical compositions either separately or in combination. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal or patch routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

Whether it is a polypeptide, peptide, or nucleic acid molecule, other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in REMINGTON'S PHARMACEUTICAL SCIENCES, 16th edition, Osol, A. (ed), 1980.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering these agents directly, they could be produced in the target cells by expression from an encoding gene introduced into the cells, e.g. in a viral vector (a variant of the VDEPT technique—see below). The vector could be targeted to the specific cells to be treated, or it could contain regulatory elements, which are switched on more or less selectively by the target cells.

Alternatively, the agent could be administered in a precursor form, for conversion to the active form by an activating agent produced in, or targeted to, the cells to be treated. This type of approach is sometimes known as ADEPT or VDEPT; the former involving targeting the activating agent to the cells by conjugation to a cell-specific antibody, while the latter involves producing the activating agent, e.g. a vaccine or fusion protein, in a vector by expression from encoding DNA in a viral vector (see for example, EP-A-415731 and WO 90/07936).

The vaccines of the present invention also include one or more adjuvant compounds. Adjuvant compounds are useful in that they enhance long term release of the vaccine by functioning as a depot. Long term exposure to the vaccine should increase the length of time the immune system is presented with the antigen for processing as well as the duration of the antibody response. The adjuvant compound also interacts with immune cells, e.g., by stimulating or modulating immune cells. Further, the adjuvant compound enhances macrophage phagocytosis after binding the vaccine as a particulate (a carrier/vehicle function).

Adjuvant compounds useful in the present invention include Complete Freund's Adjuvant (CFA); Incomplete Freund's Adjuvant (IFA); Montanide ISA (incomplete seppic adjuvant); Ribi Adjuvant System (RAS); TiterMax; Syntex Adjuvant Formulation (SAF); Aluminum Salt Adjuvants; Nitrocellulose-adsorbed antigen; Encapsulated or entrapped antigens; Immune-stimulating complexes (ISCOMs); and Gerbu^{R} adjuvant.

The invention will be further illustrated in the following non-limiting examples.

### EXAMPLES

### Example 1

### Survival of Femal A/J Mice

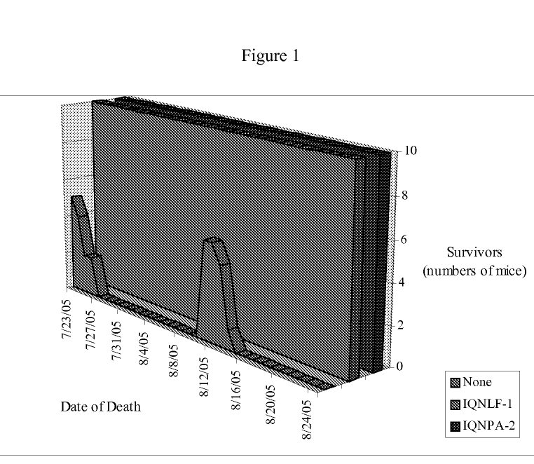

Human anthrax toxin neutralizing monoclonal antibodies previously isolated and developed have been shown to confer prophylactic and therapeutic protection 36-48 hours post challenge (mean time to death for untreated animals is 72 hours) in a mouse anthrax spore challenge model. Mice (n=10 per group) were passively protected with a single 180 μl dose of either IQNPA-2 or IQNLF-1 antibodies 2.5 hr prior to challenge with roughly 4.8×10⁵ B. anthracis Sterne strain spores. During the second challenge event the mice were not treated with antibodies but did receive 8.3×10⁵ spores. The control group during each challenge contained 5 mice. FIG. 1 shows that mice, when injected with antibodies immediately prior to challenge, were fully protected with a survival rate of 100% when challenged at day 0 and day 20.

### Example 2

### Mouse Anti-PA and Anti-LF IgG Titer Response

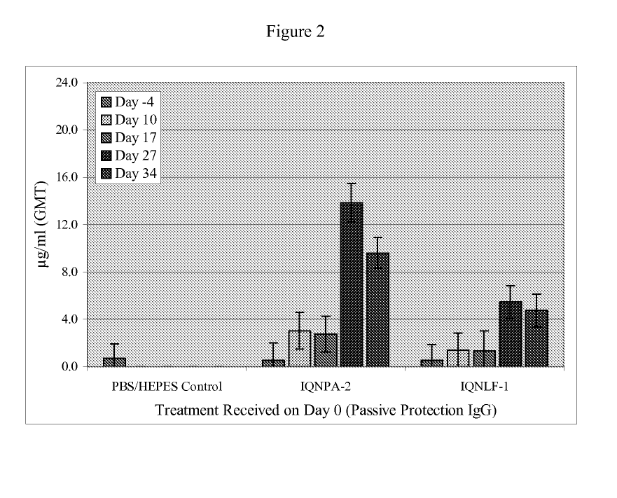

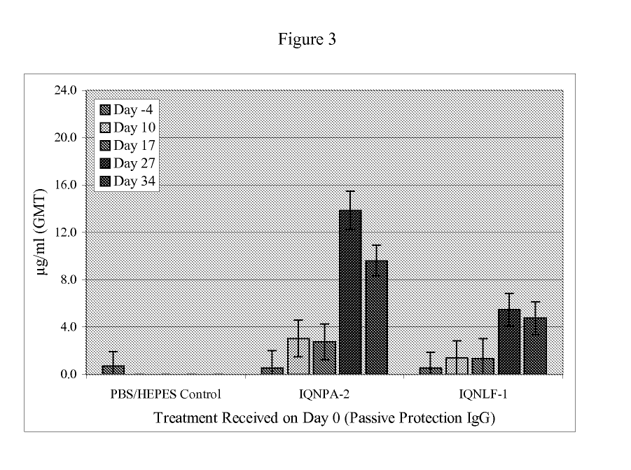

Analysis of the mouse antibody response following infection with B. anthracis Sterne strain spores showed that the animals had mounted PA and LF specific IgG responses. Mice were treated with either IQNPA-2 (n=10) or IQNLF-1 (n=10) 2.5 hr prior to an initial day 0 challenge of 4.8×10⁵ spores per mouse. Twenty days later the mice were re-challenged with 8.3×10⁵ spores per mouse. Serum samples were collected on days −4, 10, 27, and 34. Anti-PA IgG titers increased after the initial challenge and were the greatest in the mice treated with IQNPA-1, as shown in FIG. 2. Mouse anti-LF IgG titers increased after the initial challenge and were greatest in the mice treated with IQNLF-1, as shown in FIG. 3. In each case the presence of the corresponding antibody enhanced the mouse's immune response to the toxin protein.

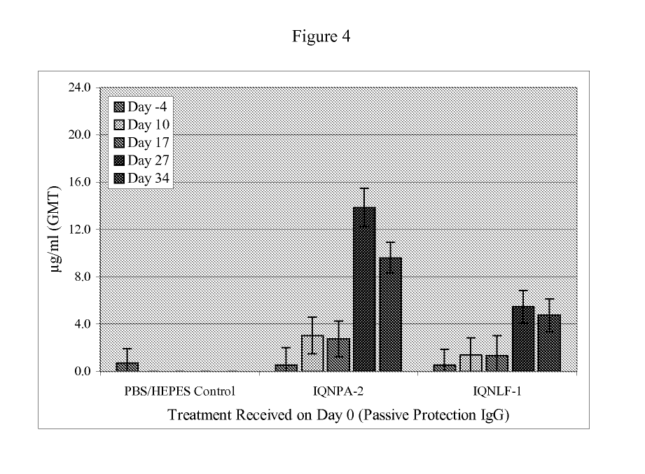

One possibility for the result is that the antibody binds to the corresponding protein and enhances uptake by antigen presenting cells either via Fc receptors or due to aggregate formation. Another possibility is that the antibodies prevent PA and LF from being rapidly internalized by macrophages via the normal lethal toxin assembly pathway and are then kept/made available for degradation via the antigen presentation route more efficiently and for a longer period of time. These antibodies possess a significant half-life, approximately 20 days (FIG. 4). IQNPA-2 has a half-life of roughly 20 days while IQNLF-1 has a reduced half-life of 15 days.

### Example 3

### Assessment of the Immune Response after Co-Administration an Anthrax Vaccine and Anti-Anthrax Antibody

Female Dunkin-Hartley guinea-pigs (300 g, 6 per group) are immunized (i.m.) at days 0, 2 and 4 with the preparations listed in Table 1. In this experiment, vaccine and antibody is pre-mixed before injection.

At time points day 0, day 8, day 14 and day 28, blood samples are drawn for anti-PA titer measurement. Titers are assessed using an ELISA specific for guinea-pig anti-PA IgG antibodies (McBride, 1998, Vaccine 16 (8), pp 810-7). The animals are challenged at day 42 (two weeks after the final vaccination) by exposure for 5 minutes to an aerosol of spores of the Ames strain of B. anthracis at a starting concentration of 10¹⁰ c.f u./mL. Spore challenge, spore preparation, recombinant PA (rPA) production and purification, measurement of absorption of rPA to alhydrogel are assessed according to the method described in McBride et al (McBride, 1998). Vaccines (0.25 human dose) are the UK human anthrax vaccine (CAMR, Porton Down) and Anthrax Vaccine Adsorbed (AVA; Bioport, US).

TABLE 1
Treatment groups, anti-PA-titers and survivors after challenge (expected results)

| | | titer × | titer × | titer × | titer × | |
|---|---|---|---|---|---|---|
| Treatment | Co-admin | 1000 | 1000 | 1000 | 1000 | survivors |
| Vaccine | IQNPA (μg) | d 0 | d 8 | d 14 | d 28 | d 56 |
| Saline | — | <1 | nd | nd | <1 | 0/6 |
| Saline | 100 | <1 | nd | nd | <1 | 0/6 |
| Alhydrogel | — | <1 | nd | nd | <1 | 0/6 |
| Alhydrogel | 100 | <1 | nd | nd | <1 | 0/6 |
| UK Human vaccine | — | <1 | 10 ± 5 | 15 ± 5 | 20 ± 5 | 1/6 |
| UK Human vaccine | 100 | <1 | 20 ± 5 | 45 ± 5 | 60 ± 10 | 2/6 |
| AVA | — | <1 | 10 ± 4 | 15 ± 5 | 15 ± 5 | 1/6 |
| AVA | 100 | <1 | 8 ± 3 | 32 ± 2 | 58 ± 3 | 2/6 |
| rPA (2.5 μg) | — | <1 | 5 ± 2 | 6 ± 1 | 6 ± 3 | 0/6 |
| rPA (2.5 μg) | 100 | <1 | 15 ± 4 | 45 ± 5 | 62 ± 8 | 2/6 |
| rPA (2.5 μg)/Alhydrogel | — | <1 | 10 ± 3 | 28 ± 10 | 34 ± 10 | 1/6 |
| rPA (2.5 μg)/Alhydrogel | 10 | <1 | 25 ± 5 | 64 ± 10 | 134 ± 10 | 6/6 |
| rPA (2.5 μg)/Alhydrogel | 100 | <1 | 18 ± 5 | 88 ± 15 | 242 ± 15 | 6/6 |
| rPA (0.025 μg)/Alhydrogel | — | <1 | 5 ± 2 | 6 ± 2 | 8 ± 2 | 0/6 |
| rPA (0.025 μg)/Alhydrogel | 100 | <1 | 10 ± 3 | 28 ± 3 | 40 ± 7 | 3/6 |
| rPA (0.25 μg)/Alhydrogel | — | <1 | 5 ± 2 | 10 ± 2 | 10 ± 2 | 1/6 |
| rPA (0.25 μg)/Alhydrogel | 100 | <1 | 15 ± 3 | 38 ± 3 | 60 ± 7 | 4/6 |
| rPA (25 μg)/Alhydrogel | — | <1 | 3 ± 1 | 12 ± 4 | 12 ± 5 | 1/6 |
| rPA (25 μg)/Alhydrogel | 100 | <1 | 4 ± 1 | 28 ± 4 | 25 ± 5 | 5/6 |

From these results we will conclude that administration of anti-PA monoclonal antibody together with an anthrax vaccine or rPA immunization results in 1) a more rapid anti-PA titer build up and 2) a significant enhancement of the anti-PA titer as compared to the immunization alone. In addition, it will also be concluded that the anti-PA antibody may to some extent replace the alhydrogel adjuvant.

### Example 4

### Assessment of the Immune Response after Successive Administration an Anthrax Vaccine and Anti-Anthrax Antibody

When the vaccine (component) and the antibody are not pre-mixed, but injected in the same site after each other, the results will be similar in that the co-administration of vaccine or rPA together with the anti-PA antibody demonstrated a marked enhancement of the rabbit anti-PA titer and a more rapid titer build up than compared to the immunizations alone.

### Example 5

### Assessment of the Immune Response after Co-Administration of an Anthrax Vaccine and Anti-Anthrax Antibody

When the co-administration is only performed during the first of four immunizations, the results will be similar in that the co-administration of vaccine or rPA together with the anti-PA antibody demonstrated a marked enhancement of the rabbit anti-PA titer and a more rapid titer build up than compared to the immunizations alone.

### Example 6

### Assessment of the Immune Response after Co-Administration of an Anthrax Vaccine and Anti-Anthrax Antibody

When the co-administration was only performed during the final of four immunizations, the results will be similar in that the co-administration of vaccine or rPA together with the anti-PA antibody demonstrated a marked enhancement of the rabbit anti-PA titer than compared to the immunizations alone.

### Example 7

### Assessment of the Immune Response after Co-Administration of an Anthrax Vaccine and Anti-Anthrax Antibody

When the co-administration is performed with recombinant LF (rLF) and the anti-LF antibody IQNLF, the results will be similar in that the co-administration of rLF together with the anti-LF antibody demonstrated a marked enhancement of the rabbit anti-LF titer and a more rapid titer build up than compared to the immunizations alone.

### Example 8

### Assessment of the Immune Response after Co-Administration of an Anthrax Vaccine and Anti-Anthrax Antibody

When the co-administration is performed with rPA+rLF and IQNPA+IQNLF, the results will be similar in that the co-administration resulted in higher titers of both anti-PA and anti-LF antibodies and a more rapid titer build up compared to the immunizations alone.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of inducing an immune response to an antigen in a subject comprising administering to said subject said antigen and an antibody or fragment thereof specific for the said antigen.

2. The method of claim 1, wherein said immune response is an antibody response.

3. The method of claim 1, wherein in said immune response is of higher magnitude than when said antigen is administered alone.

4. The method of claim 1, wherein said antigen is administered in the form of a vaccine.

5. The method of claim 4, wherein said vaccine is a protein based vaccine or a DNA based vaccine.

6. The method of claim 1, wherein said antigen is a pathogen or immunogenic component thereof.

7. The method of claim 6, wherein said pathogen is a toxin, a virus, a bacterium, a fungus, a protozoan, a mycloplasma, a rickettsia or a parasite.

8. The method of claim 7, wherein said bacterium is Bacillus anthracis.

9. The method of claim 1, wherein said antigen is a Bacillus anthracis protective antigen polypeptide of a Bacillus anthracis lethal factor polypeptide and said antibody is IQNPA or IQNLF.

10. The method of claim 1, wherein said antigen and said antibody are administered post exposure to anthrax

11. The method of claim 1, wherein said antigen and antibody are administered concurrently.

12. The method of claim 1, wherein said antibody is administered prior to said antigen.

13. The method of claim 1, wherein said antigen is administered prior to said antibody.

14. The method of claim 4, wherein said vaccine is AVA.

15. A method of enhancing antigen presentation of an antigen comprising contacting an antigen presenting cell with said antigen and an antibody or fragment thereof specific for the said antigen.

16. The method of claim 15, wherein said antigen is a pathogen or immunogenic component thereof.

17. The method of claim 16, wherein said pathogen is a toxin, a virus, a bacterium, a fungus, a protozoan, a mycloplasma, a rickettsia or a parasite.

18. The method of claim 17, wherein said bacterium is Bacillus anthracis.

19. The method of claim 15, wherein said antigen is a Bacillus anthracis protective antigen polypeptide or a Bacillus anthracis lethal factor polypeptide and said antibody is IQNPA or IQNLF.

20. A composition comprising monoclonal antibody IQNPA or IQNLF and an anthrax vaccine.

21. A composition comprising monoclonal antibody IQNPA and IQNLF and an anthrax vaccine.

22. The composition of claim 20 or 21, wherein said anthrax vaccine is AVA.

23. A method for increasing the time an antigen to be in circulation longer than in a subject comprising administering to said subject said antigen and an antibody or fragment thereof specific for the said antigen.

24. The method of claim 23, wherein said antigen is a pathogen or immunogenic component thereof.

25. The method of claim 24, wherein said pathogen is a toxin, a virus, a bacterium, a fungus, a protozoan, a mycloplasma, a rickettsia or a parasite.

26. The method of claim 25, wherein said bacterium is Bacillus anthracis.

27. The method of claim 23, wherein said antigen is a Bacillus anthracis protective antigen polypeptide or a Bacillus anthracis lethal factor polypeptide and said antibody is IQNPA or IQNLF.
